(19) United States
(12) Patent Application Publication
(10) Pub. No.: US 2006/0276742 A1
(43) Pub. Date: Dec. 7, 2006

(54) Iontophoresis device and method of controlling the same

(75) Inventors: Akihiko Matsumura, Shibuya-ku (JP); Takehiko Matsumura, Shibuya-ku (JP); Mizuo Nakayama, Shibuya-ku (JP); Hidero Akiyama, Shibuya-ku (JP)

Correspondence Address: SEED INTELLECTUAL PROPERTY LAW GROUP PLLC, 701 FIFTH AVE, SUITE 6300, SEATTLE, WA, 98104-7092, US

(73) Assignee: Transcutaneous Technologies, Inc., Shibuya-ku (JP)

(21) Appl. No.: 11/443,564

(22) Filed: May 30, 2006

(30) Foreign Application Priority Data
Jun. 2, 2005 (JP) 2005-163002

(51) Int. Cl. A61N 1/30

(52) U.S. Cl. 604/20

(57) ABSTRACT

An iontophoresis device, and a control method thereof, may be capable of increasing movement speed of a drug into a living body and/or may be capable of administering a drug having a high molecular weight. The iontophoresis device may include an active electrode assembly comprising: a first electrode; a drug holding part that receives a current from the first electrode; and a first ion exchange membrane that selectively passes ions of a first polarity, the first ion exchange membrane being placed on a front side of the drug holding part. Drug ions of the first polarity generated by the dissociation of a drug held in the drug holding part may be administered to a living body through the first ion exchange membrane in contact with skin of the living body. The iontophoresis device may include heating means to heat the skin in contact with the first ion exchange membrane.

### BACKGROUND OF THE INVENTION

1. Field of the Invention

The present disclosure relates to an iontophoresis device for administering drug ions to a living body and a control method thereof.

2. Description of the Related Art

A conventional transdermal administration method of allowing a drug to permeate into skin by applying the drug to the skin exists. However, movement of the drug by this method occurs mainly through diffusion based on a concentration gradient, so the amount of the drug administered per unit time (administration speed of the drug) is small.

Iontophoresis is known as a method of enhancing the administration speed of a drug. As shown in FIG. 5, an iontophoresis device may include a active electrode assembly 10 having a drug holding part 14 that holds a drug solution, and a counter electrode assembly 20 as a counterpart to the active electrode assembly 10, and the drug ions are electrically driven to be transmitted into a living body by applying a voltage of the same polarity as that of drug ions in the drug holding part 14 to the active electrode assembly 10 under the condition of keeping the drug solution in contact with skin A. In FIG. 5, reference numerals 11 and 21 denote electrodes, 22 denotes an electrolyte solution for maintaining conductivity between the electrode 21 and the skin A, and 30 denotes a power source.

Iontophoresis may have advantages such as the following: little pain caused to a living body; drugs may be administered without the first pass effect; and the administered amount of a drug can be controlled by the amount of electric current used. Iontophoresis is considered to be an excellent drug administration method that be substituted for injection and/or oral administration.

However, when using the iontophoresis device shown in FIG. 5, drugs that can be administered at a current level where skin is not damaged are limited to those having relatively small molecular weights. Furthermore, even drugs having low molecular weights may not be able to be administered effectively within a set allowable period of time for administration. A need thus exists for an increase in drug administration speed during iontophoresis.

JP 06-070987 A discloses a method of increasing the administration speed of a drug using iontophoresis.

FIG. 6 illustrates the configuration of an iontophoresis device disclosed in JP 06-070987 A. Electrodes 7a and 7b, which apply positive and negative voltage, respectively, and ultrasonic elements 8a and 8b, which transmit ultrasonic energy to skin A, are housed in containers 4a and 4b, into which drug solutions are injected.

According to JP 06-070987 A, in the iontophoresis device above, the skin A receives ultrasonic energy from the ultrasonic elements 8a and 8b to decrease physical resistance, whereby it becomes easier to transfer the drug into the skin. Furthermore, any keratinocytes present and substantially peeling from the surface of the skin A may be removed, thus lowering the overall resistance and allowing the drug to be administered stably at a low current.

However, experiments performed the applicants of the present invention tend to show that no significant increase in an administration speed of a drug is seen when ultrasonic waves are applied in the iontophoresis device shown in FIG. 6.

### BRIEF SUMMARY OF THE INVENTION

In at least one embodiment an iontophoresis device, and a control method thereof, may be capable of remarkably increasing an administration speed of a drug.

In at least one embodiment an iontophoresis device, and a control method thereof, may be capable of administering a drug having a higher molecular weight into a living body.

According to one aspect, an iontophoresis device may include an active electrode assembly comprising:

a first electrode;

a drug holding part that holds a drug and to which an electrical potential of a first polarity is applied via the first electrode, drug ions of the first polarity being generated by dissociation of the drug held in the drug holding part; and

a first ion exchange membrane that selectively passes ions of the first polarity, the first ion exchange membrane being placed on a front side of the drug holding part, the drug ions of the first polarity being administered to a living body through the first ion exchange membrane in contact with skin of the living body; and

a heating means for heating the skin of the living body in contact with the first ion exchange membrane.

In a method of controlling an iontophoresis device that includes an active electrode assembly comprising:

a first electrode;

a drug holding part that holds a drug and to which an electrical potential of a first polarity is applied via the first electrode, drug ions of the first polarity being generated by dissociation of the drug held in the drug holding part; and

a first ion exchange membrane that selectively passes ions of the first polarity, the first ion exchange membrane being placed on a front side of the drug holding part, the drug ions of the first polarity being administered to a living body through the first ion exchange membrane in contact with skin of the living body; and

a heating means;

the method comprising applying a voltage of the first polarity to the first electrode while dissipating heat from the heating means to heat the skin of the living body in contact with the first ion exchange membrane.

More specifically, when delivering drug ions using an iontophoresis having an ion exchange membrane (the ion exchange membrane selectively passing ions of the same polarity as that of the drug) interposed between the skin of a living body and a drug solution containing the drug ions, the skin of the living body contacting the ion exchange membrane may be heated, preferably to about 39° C. to 42° C., thus remarkably increasing the speed at which the drug ions are administered to the living body. Even drugs having a high molecular weight, which are not administered using conventional iontophoresis may be administered to the living body at sufficient speed.

That the administration speed of a drug increases when the skin surface into which the drug is administered by iontophoresis is heated may appear to be a natural effect, owing to activation of the skin surface, for example.

However, no significant change occurs in drug administration speed when heating the skin and using an ordinary iontophoresis device, such as that shown in FIG. 5, which does not include an ion exchange membrane that selectively passes ions of the same polarity as that of drug ions to be administered.

It is not completely clear why the drug administration speed when heating the skin surface is observed only in connection with the iontophoresis device that administers the drug ions through an ion exchange membrane interposed between the skin and the drug solution, not is it completely clear why this is not observed when an iontophoresis device that does not include an ion exchange membrane.

However, the ratio of a drug moving to a living body through gaps between keratinocytes may be much larger for the iontophoresis device with an ion exchange membrane between the skin and the drug solution than for the iontophoresis device without an ion exchange membrane similarly placed. The inventors of the present invention presume that the ratio difference may be an important factor for explaining the increased drug administration speed only when using the iontophoresis device having an ion exchange membrane between the skin the drug solution.

More specifically, according to studies by Grimnes, S. “Pathways of Ionic Flow Through Human Skin in vivo,” ActaDerm. Venereol. (Stockh.) 64, 93-98(1984); Burnette, R. R. Iontophoresis. In J. Hadgraft and R. H. Guy (Eds) (1988); Marcel Dekker, Transdermal Drug Delivery, New York, 1988, Ch. 1; Siddiqui, O., Sun, Y., Liu, J. C. and Chien, Y. W. “Facilitated transport of insulin,” J. Pharm. Sci. 76, 341-345, (1987); and Yoshida, N. H. and Roberts, M. S., “Structure-Transport Relationships in Transdermal Iontophoresis,” Adv. Drug Deliv. Rev. 9, 239-264, (1992), the following has been observed: the main movement path of a drug to a living body in an iontophoresis device that does not include an ion exchange membrane is through cutaneous appendages such as sweat glands, sebaceous glands, or apocrine glands, while the movement amount of a drug through gaps between keratinocytes is negligibly small.

Thus, if any increase of the administration speed occurs in an iontophoresis device of the type studied, without an ion exchange membrane between the skin and the drug solution, it can be ascribed to the change in cutaneous appendages by heating. However, as described above, no significant change occurs in drug administration speed with this type of iontophoresis device. Therefore the change in cutaneous appendages by heating is considered not to have a large influence on the administration speed of a drug.

In contrast, the ratio of a drug moving to a living body through gaps between keratinocytes is considered to be much larger when using the iontophoresis device having an ion exchange membrane between the skin and the drug solution. Further, the skin temperature (about 39° C.) at which the administration speed of a drug increases in the present invention matches the temperature at which the gaps of keratinocytes enlarge greatly. Therefore, the increase in the administration speed of a drug by heating can be considered to be a phenomenon peculiar to iontophoresis devices where the ratio of a drug transferred through gaps between keratinocytes is equal to or greater than a predetermined value.

An arbitrary ion exchange membrane that selectively passes ions of a first polarity and inhibits or suppresses the passage of ions of a second polarity can be used as a first ion exchange membrane in the present invention. Preferably, an ion exchange membrane can be used in which a portion, or the entirety, of pores of a porous film contain an ion exchange resin having an exchange group whose counter ions are those of the first polarity introduced thereto.

It is possible to use as heating means of the present invention arbitrary means capable of heating the skin of a living body with which the first ion exchange membrane comes into contact preferably to about 39° C. to 42° C. Preferably, a flexible sheet shape heating element that can flexibly follow the curved surface of the skin of a living body or the movement of the living body is used, such as a rubber heater or an infrared radiator capable of heating the skin of a living body under a non-contact condition.

The term “drug” as used herein refers to substances that have a certain pharmacological effect and are applied to a living body for the purpose of treatment, recovery, or prevention of disease, or to promote or maintain of health, irrespective of whether or not the drug is prepared (formulated). The term “drug ions” refers to ions generated by the dissociation of a drug and having a pharmacological function. The dissociation of a drug into drug ions may occur when the drug is dissolved in a solvent such as water, an acid, or an alkali, or may occur owing to the application of a voltage, the addition of an ionization agent, or the like.

The active electrode assembly may further include a first electrolyte solution holding part that holds an electrolyte solution in contact with the first electrode, and a second ion exchange membrane that selectively passes ions of a second polarity, the second ion exchange membrane being placed on a front side of the first electrolyte solution holding part (a side closer to the skin), the drug holding part being placed on a front side of the second ion exchange membrane. This configuration may help to prevent the decomposition of drug ions in the vicinity of the first electrode. Furthermore, this configuration may help to prevent variations in pH that may occur at the skin interface due to the movement of H⁺ ions and OH⁻ ions that are generated in the first electrolyte holding part to the drug holding part, inflammation that may occur on the skin surface, and the like. The drug may thus be administered with enhanced stability and safety.

The iontophoresis device of the present invention may further include a counter electrode assembly having: a second electrode; a second electrolyte solution holding part that holds an electrolyte solution in contact with the second electrode; a third ion exchange membrane that selectively passes ions of the first polarity, the third ion exchange membrane being placed on a front side of the second electrolyte solution holding part; a third electrolyte solution holding part that holds an electrolyte solution, the third electrolyte solution holding part being placed on a front side of the third ion exchange membrane; and a fourth ion exchange membrane that selectively passes ions of the second polarity, the fourth ion exchange membrane being placed on the front side of the third electrolyte solution holding part. This configuration may help to reduce variations in a pH value at the interface between the counter electrode assembly and the skin, inflammation that may occur on the skin surface, and the like. The drug may thus be administered with enhanced stability and safety.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIGS. 1A and 1B illustrate a configuration of an iontophoresis device according to the present invention;

FIG. 2 illustrates a usage of an iontophoresis device according to an embodiment of the present invention;

FIG. 3A illustrates an embodiment of an active electrode assembly;

FIG. 3B illustrates an embodiment of a counter electrode assembly;

FIG. 4 illustrates a configuration of an iontophoresis device of an embodiment according to the present invention;

FIG. 5 illustrates a configuration of a conventional iontophoresis device; and

FIG. 6 illustrates a configuration of a conventional iontophoresis device.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

The headings provided herein are for convenience only and do not interpret the scope or meaning of the embodiments.

An iontophoresis device that administers a drug whose active ingredient dissociates into positive drug ions (e.g. lidocaine hydrochloride or morphine hydrochloride as an anesthetic) is used as an example in the following embodiment. An iontophoresis device that administers a drug whose active ingredient dissociates into negative drug ions (e.g., ascorbic acid as a vitamin agent) can be configured by inverting the polarity of a voltage applied to electrodes of the iontophoresis device, and inverting the polarity (positive vs. negative) of an exchange group introduced to an ion exchange membrane or an ion exchange resin.

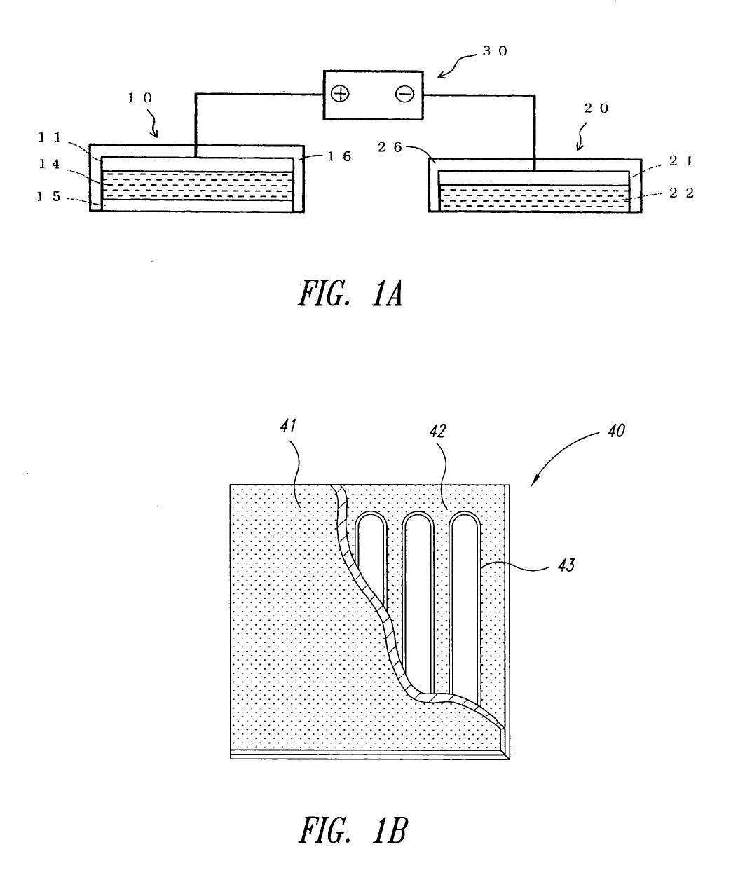

FIG. 1A illustrates portions of an active electrode assembly 10, a counter electrode assembly 20, and a power source 30 that form an iontophoresis device 1 of the present invention. FIG. 1B illustrates a partially cut-away view showing a portion of a rubber heater 40 of the iontophoresis device 1.

Referring to FIG. 1A, the active electrode assembly 10 comprises an electrode 11, a drug holding part 14 that holds a drug solution in contact with the electrode 11 to receive a current or electrical potential from the electrode 11, a cation exchange membrane 15 placed on a front side of the drug holding part 14, and a container 16 that contains the components 11, 14, 15, and 16. The counter electrode assembly 20 comprises an electrode 21, an electrolyte solution holding part 22 that holds an electrolyte solution in contact with the electrode 21 to receive a current from the electrode 21, and a container 26 that contains the components 21, 22, and 26.

The electrodes 11 and 21 may use arbitrary conductive materials, without any specific limitations placed thereon. Active electrodes such as silver/silver-chloride electrodes capable of suppressing the generation of H⁺ ions and OH⁻ ions due to the electrolysis of water may also be used.

The drug holding part 14 holds a drug solution whose active ingredient dissociates into positive drug ions by dissolution.

The electrolyte solution holding part 22 holds an electrolyte solution that helps to ensure the passage of a current. Phosphate buffered saline or physiological saline can be used as the electrolyte solution. Alternatively, electrolysis of water may be prevented by using an electrolyte that is more readily oxidized or reduced compared than water undergoes electrolysis, (oxidation at a positive electrode and reduction at a negative electrode). For example, use of an inorganic compound such as ferrous sulfate or ferric sulfate, a medical agent such as ascorbic acid (vitamin C) or sodium ascorbate, an organic acid such as lactic acid, oxalic acid, malic acid, succinic acid, or fumaric acid, and/or a salt thereof or a mixture thereof, may prevent the electrolysis of water. The generation of gas, increases in conductive resistance due to the generation of gas, and variations in pH may thus be prevented.

The drug holding part 14 and the electrolyte solution holding part 22 may hold a drug solution or an electrolyte solution in a liquid state. Alternatively, it is also possible to impregnate a carrier with the drug solution or the electrolyte solution. The carrier may be made of an arbitrary material having a water absorbing property, such as a fibrous sheet (gauze, a filter paper, etc.) or a polymer gel sheet, such as a hydrogel of an acrylic resin or segmented polyurethane gel. In this case, handling characteristics and the like may be enhanced.

The impregnation ratio of the drug solution or electrolyte solution in the carrier should be set to an appropriate value, at which sufficient passage of current and a sufficient transport number may be obtained. By setting the impregnation ratio of the drug solution to be from 20% to 60% in the drug holding part 14, a high transport number (high drug delivery) equal to or greater than 70% can be obtained, for example.

The impregnation ratio herein is represented in wt %, i.e., 100×(W−D)/D [%], where D is dry state weight and W is a weight after impregnation. The transport number refers to the ratio of current that contributes to the movement of drug ions into a living body to the entire current supplied to the active electrode assembly.

The cation exchange membrane 15 functions to selectively pass positive ions. For example, a cation exchange membrane such as NEOSEPTA CM-1, CM-2, CMX, CMS, or CMB manufactured by Tokuyama Corp. may be used. In particular, it may be preferable to use a cation exchange membrane in which a portion of, or the entirety of, pores in a porous film made of polyolefin resin, vinyl chloride resin, fluorine resin, polyamide resin, polyimide resin, or the like are filled with a cation exchange resin. The cation exchange resin may be loaded by employing the following method, for example: impregnating the pores of the porous film with a solution in which a polymerization initiator is mixed with a cross-linking monomer, such as styrene-divinyl benzene or chloromethyl styrene-divinyl benzene; polymerizing the resultant; and introducing a cation exchange group such as a sulfonic group, a carboxylic acid group, or a phosphonic acid group into the polymer.

A battery, a constant voltage device, a constant current device, a constant voltage/constant current device, or the like may be used as the power source 30. It is preferable to use constant current device in which current adjustment can be performed in a range from 0.01 to 1.0 mA/cm², preferably from 0.01 to 0.5 mA/cm², and which operates under a safe voltage condition of 50 V or less, preferably 30 V or less.

Referring to FIG. 1B, the rubber heater 40 comprises a configuration in which two rubber sheets 41 and 42 made of a flexible material, such as silicon rubber or urethane rubber, are laminated together with a resistance heating line 43 connected to a power source (not shown).

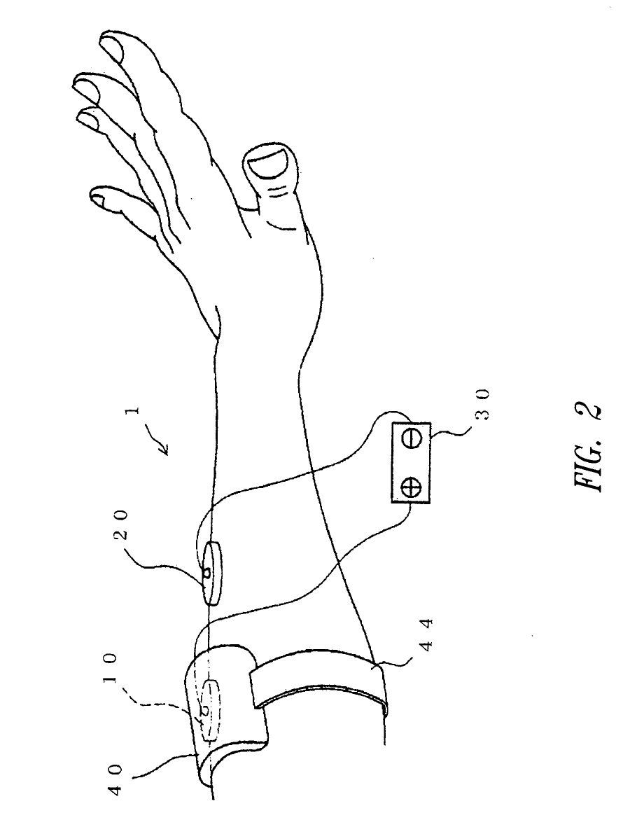

FIG. 2 illustrates the usage of the iontophoresis device 1.

As shown in FIG. 2, the rubber heater 40 is placed so as to cover the active electrode assembly 10 in contact with the skin of a living body. Reference numeral 44 denotes a belt that fixes the rubber heater 40. The counter electrode assembly 20 is placed on the skin of a living body at a position away from the active electrode assembly 10.

When administering a drug, a positive voltage is applied to the electrode 11 from the power source 30, and a negative voltage is applied to the electrode 21 from the power source 30. Drug ions in the drug holding part 14 are thus driven toward the living body. Furthermore, by passing a current to the resistance heating line 43 during the application of the voltages to the electrodes 11 and 21, the rubber heater 40 generates heat. The skin surface contacting the cation exchange membrane 15 is heated to an appropriate temperature (preferably from 39° C. to 42° C.), thus enlarging gaps between keratinocytes on the skin surface and promoting the smooth movement of drug ions to the living body.

A temperature sensor may be provided on the rubber heater 40, the active electrode assembly 10, or the skin surface. Feedback control may then be performed to keep the temperature of the skin surface within the an appropriate temperature range.

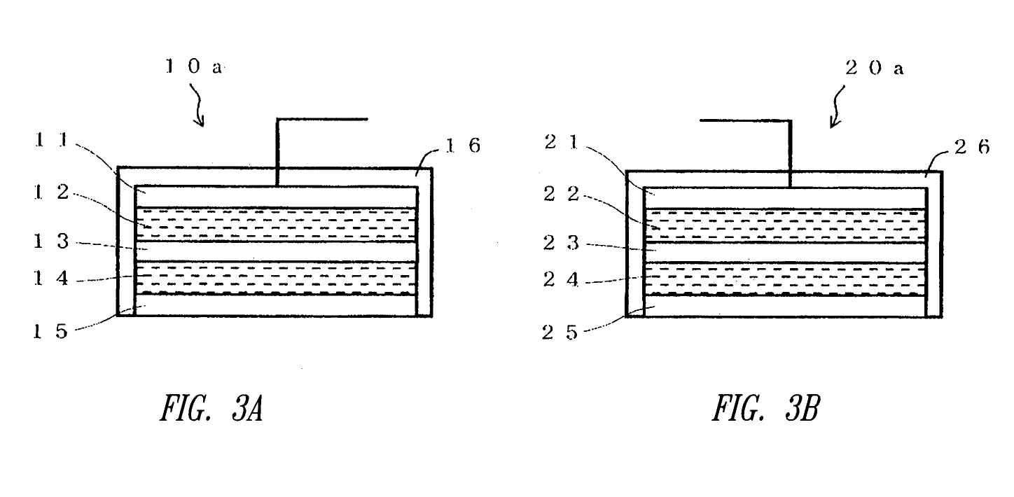

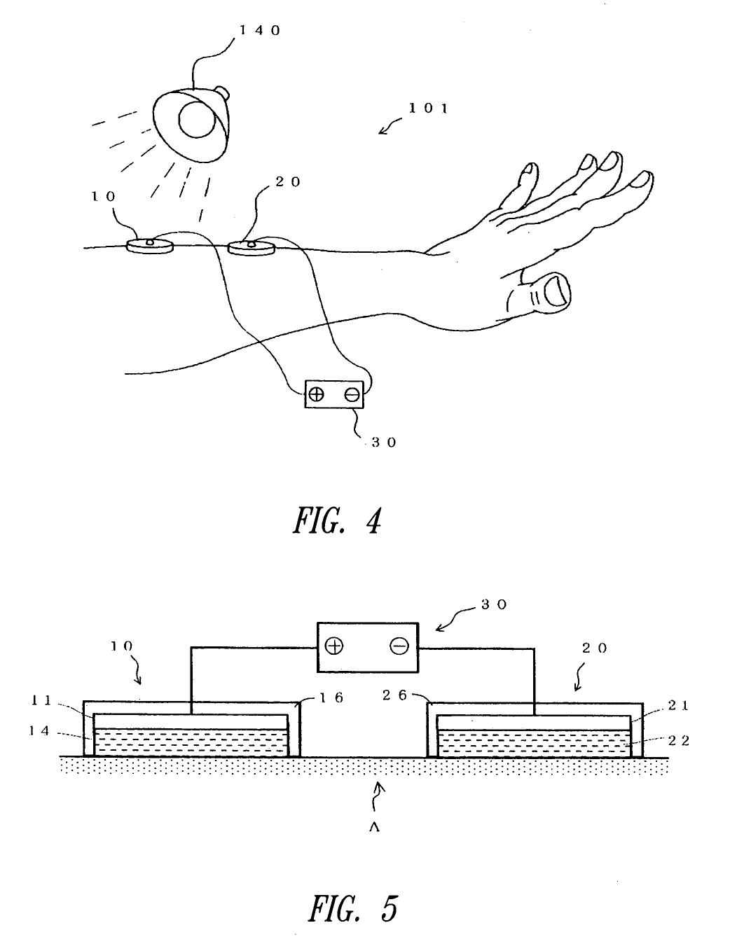

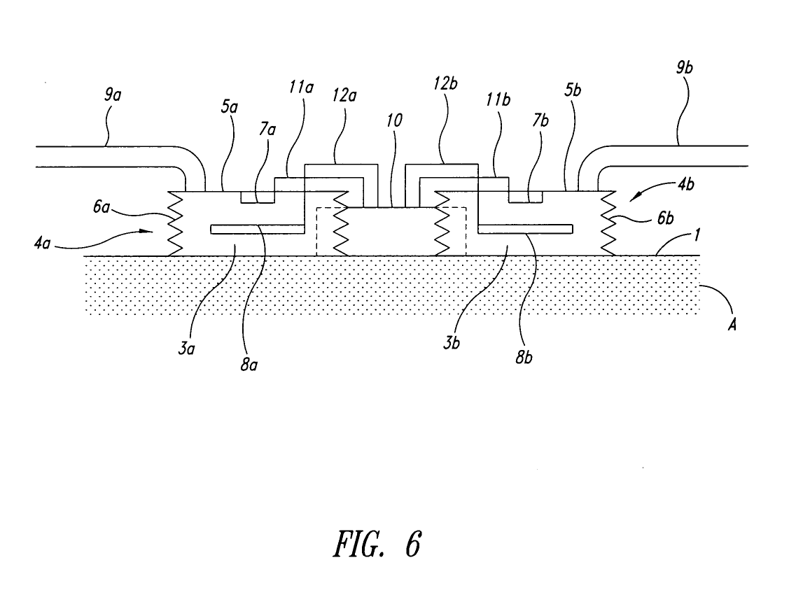

FIG. 3A illustrates a configuration of an active electrode assembly 10a that can be used for the iontophoresis device of the present invention in place of the active electrode assembly 10.

The active electrode assembly 10a comprises an electrolyte solution holding part 12 and an anion exchange membrane 13 on a front side of the electrode 11. In addition, the active electrode assembly 10a comprises the drug holding part 14, and the cation exchange membrane 15. The passage of current from the electrode 11 to the drug holding part 14 is performed through the electrolyte solution holding part 12 and the anion exchange membrane 13.

The electrolyte solution holding part 12 holds an electrolyte solution in contact with the electrode 11 to receive a current from the electrode 11, and holds the electrolyte solution in a manner similar to that of the electrolyte solution holding part 22.

The anion exchange membrane 13 functions to selectively pass negative ions. For example, an anion exchange membrane such as NEOSEPTA AM-1, AM-3, AMX, AHA, ACH, or ACS manufactured by Tokuyama Corp. may be used. In particular, it may be preferable to use an anion exchange membrane in which a portion of, or the entirety of, pores in a porous film made of polyolefin resin, vinyl chloride resin, fluorine resin, polyamide resin, polyimide resin, or the like are filled with an anion exchange resin. The anion exchange resin may be loaded by employing the following method, for example: impregnating the pores of the porous film with a solution in which a polymerization initiator is mixed with a cross-linking monomer such as styrene-divinyl benzene or chloromethyl styrene-divinyl benzene; polymerizing the resultant; and introducing an anion exchange group such as any one of primary to tertiary amino groups, a quarternary ammonium group, a pyridyl group, an imidazole group, a quaternary pyridinium group, or a quaternary imidazolium group into the polymer.

The iontophoresis device having the active electrode assembly 10a in place of the active electrode assembly 10 may be used in the manner shown in FIG. 2 in the same way as in the iontophoresis device 1 to administer drug ions from the skin surface to the living body at a high speed, through gaps between keratinocytes that enlarge due to the heating by the rubber heater 40.

In addition, the movement of the drug ions to the electrolyte solution holding part 12 and the movement of H⁺ ions generated by electrolysis at the electrode 11 to the drug holding part 14 may be inhibited by the anion exchange membrane 13. Consequently, the dissolution of a drug at the electrode 11 and variations in pH on the skin interface may be suppressed. The stability and safety of the administration of a drug can thus be enhanced.

Furthermore, the drug holding part 14 is separated from the electrolyte solution holding part 12. The generation of gas and variation s in pH may therefore be suppressed by mixing a material having an oxidation-reduction potential less than that of water into the electrolyte solution of the holding part 12, or by using a buffer solution in which a plurality of ions are present. Carbon or an inactive metal such as platinum may be used in the electrodes 11 and 21 in this case. In particular, a composite carbon electrode comprising a terminal member, where carbon powder is mixed into a polymer matrix, and a conductive sheet made of carbon fibers or carbon fiber paper may be used. The carbon fibers or carbon fiber paper may be impregnated with a polymer elastomer. The composite carbon electrode has high conductivity and flexibility. In addition, the composite carbon electrode may prevent metal ions from moving into the living body.

FIG. 3B illustrates a counter electrode assembly 20a that may be used for the iontophoresis device of the present invention in place of the counter electrode assembly 20.

The counter electrode assembly 20a comprises a cation exchange membrane 23 placed on a front side of the electrolyte solution holding part 22, an electrolyte solution holding part 24 placed on a front side of the cation exchange membrane 23, and an anion exchange membrane 25 placed on a front side of the electrolyte solution holding part 24. The counter electrode assembly 20a further comprises the electrode 21 and the electrolyte solution holding part 22.

The electrolyte solution holding part 24 may be configured in the same manner as the electrolyte solution holding part 22. Further, the cation exchange membrane 23 and the anion exchange membrane 25 can be configured in the same way as the cation exchange membrane 15 and the anion exchange membrane 13, respectively.

The iontophoresis device having the counter electrode assembly 20a in place of the counter electrode assembly 20 may be used in the manner shown in FIG. 2, similar to the iontophoresis device 1. The iontophoresis device may administer drug ions to the living body at high speed through the skin surface, at which the gaps between keratinocytes enlarge due to heating by the rubber heater 40.

In addition, the movement of OH⁻ ions generated by electrolysis at the electrode 21 to the living body interface may be inhibited by the cation exchange membrane 23. Consequently, the dissolution of a drug at the electrode 21 and variations in pH on the skin interface may be suppressed. The stability and safety of the administration of a drug can thus be enhanced

FIG. 4 illustrates an iontophoresis device 101 of another embodiment.

The iontophoresis device 101 has a similar configuration as that of the iontophoresis device 1, and comprises an infrared heater 140 in place of the rubber heater 40. It may be possible to use the active electrode assembly 10a and/or the counter electrode assembly 20a in place of the active electrode assembly 10 and/or the counter electrode assembly 20.

In the iontophoresis device 101, drug ions in the drug holding part 14 are driven to the living body when power is applied from the power source 30. Furthermore, the skin surface contacting the cation exchange membrane 15 may be heated to an appropriate temperature (preferably to about 39° C. to 42° C.), and gaps between keratinocytes on the skin surface may enlarge, when infrared light is emitted from the infrared heater 140 during the administration of a drug. Smooth movement of the drug ions into the living body can thus be promoted.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis devices generally described above.

Some embodiments may have substitutive structure. For example, cases of using the rubber heater 40 or the infrared heater 140 as heating means have been described herein. Any other arbitrary contact type heating means, such as a heating tool containing warm water in a flexible bag container, and any other arbitrary non-contact type heating means, such as a hot air heater or an ultrasonic vibrating element, may be used in place of the rubber heater 40 or the infrared heater 140, provided that the heating element is capable of heating the skin of a living body contacting the cation exchange membrane to an appropriate temperature (from 39° C. to 42° C.) and can promote the safety of the living body.

Some embodiments may have additional structure. For example, a porous separation film having pores with a size capable of suppressing the passage of: electrolyte molecules or first or second conductive ions generated by the dissociation of the electrolyte molecules in the electrolyte solution holding part 12; or drug molecules or drug ions in the drug holding part 14, may be placed between the electrolyte solution holding part 12 and the drug holding part 14 in the active electrode assembly 10a. This may promote stability in solution quality over a long period of time. It is also possible to form a layer of a water soluble polymer on a front side of the cation exchange membrane 15 of the working electrode structure 10 or 10a, or on a front side of the anion exchange membrane 25 of the counter electrode assembly 20a. The drug administration speed may thus be increased. In each case, the promotion of the smooth movement of a drug to a living body by enlarging gaps between keratinocytes by heating may be achieved. Iontophoresis devices having such configurations thus fall under the scope of the present invention.

Further, it is possible to omit the electrolyte solution holding part 22 and/or the container 26 of the counter electrode assembly 20. It is also possible to omit the counter electrode assembly from the iontophoresis device because the drug can be administered by applying a voltage to the working electrode structure under the condition that the active electrode assembly is in contact with the living body and a part of the living body is in contact with an electrical earth member. The degree of satisfactory contact between the counter electrode assembly and the living body may be inferior to that of the above embodiments in this case. However, the promotion of smooth movement of a drug to the living body by enlarging the gaps of keratinocytes by heating may still be achieved.

Furthermore, although cases where the active electrode assembly, counter electrode assembly, electrode, and heating member comprise separate members have been described, it may also possible to enhance handleability by incorporating parts thereof, or the entirety thereof, into a single casing.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: JP 06-070987 A.

Aspects of the various embodiments can be modified, if necessary, to employ systems, circuits and concepts of the various patents, applications and publications to provide yet further embodiments.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

1. An iontophoresis device, comprising:
an active electrode assembly comprising:
a first electrode;
a drug holding part that holds a drug and to which an electrical potential of a first polarity is applied via the first electrode, drug ions of the first polarity being generated by dissociation of the drug held in the drug holding part; and
a first ion exchange membrane that selectively passes ions of the first polarity, the first ion exchange membrane being placed on a front side of the drug holding part, the drug ions of the first polarity being administered to a living body through the first ion exchange membrane in contact with skin of the living body; and
a heating means for heating the skin of the living body in contact with the first ion exchange membrane.

2. An iontophoresis device according to claim 1, wherein the heating means comprises a rubber heater.

3. An iontophoresis device according to claim 1, wherein the heating means comprises an infrared radiator.

4. An iontophoresis device according to claim 1, wherein the active electrode assembly further comprises:
a first electrolyte solution holding part that holds an electrolyte solution in contact with the first electrode; and
a second ion exchange membrane that selectively passes ions of a second polarity, the second ion exchange membrane being placed on a front side of the first electrolyte solution holding part,
wherein the drug holding part is placed on a front side of the second ion exchange membrane.

5. An iontophoresis device according to claim 1, further comprising a counter electrode assembly comprising:
a second electrode;
a second electrolyte solution holding part that holds an electrolyte solution in contact with the second electrode;
a third ion exchange membrane that selectively passes ions of the first polarity, the third ion exchange membrane being placed on a front side of the second electrolyte solution holding part;
a third electrolyte solution holding part that holds an electrolyte solution, the third electrolyte solution holding part being placed on a front side of the third ion exchange membrane; and
a fourth ion exchange membrane that selectively passes ions of the second polarity, the fourth ion exchange membrane being placed on a front side of the third electrolyte solution holding part.

6. A method of controlling an iontophoresis device, comprising:
a first electrode;
a drug holding part that holds a drug and to which an electrical potential of a first polarity is applied via the first electrode, drug ions of the first polarity being generated by dissociation of the drug held in the drug holding part; and
a first ion exchange membrane that selectively passes ions of the first polarity, the first ion exchange membrane being placed on a front side of the drug holding part, the drug ions of the first polarity being administered to a living body through the first ion exchange membrane in contact with skin of the living body; and
a heating means;
the method comprising applying a voltage of the first polarity to the first electrode while dissipating heat from the heating means to heat the skin of the living body in contact with the first ion exchange membrane.

7. An iontophoresis device according to claim 2 wherein the active electrode assembly further comprises:
a first electrolyte solution holding part that holds an electrolyte solution in contact with the first electrode; and
a second ion exchange membrane that selectively passes ions of a second polarity, the second ion exchange membrane being placed on a front side of the first electrolyte solution holding part,
wherein the drug holding part is placed on a front side of the second ion exchange membrane.

8. An iontophoresis device according to claim 2 further comprising a counter electrode assembly comprising:
a second electrode;
a second electrolyte solution holding part that holds an electrolyte solution in contact with the second electrode;
a third ion exchange membrane that selectively passes ions of the first polarity, the third ion exchange membrane being placed on a front side of the second electrolyte solution holding part;
a third electrolyte solution holding part that holds an electrolyte solution, the third electrolyte solution holding part being placed on a front side of the third ion exchange membrane; and
a fourth ion exchange membrane that selectively passes ions of the second polarity, the fourth ion exchange membrane being placed on a front side of the third electrolyte solution holding part.

9. An iontophoresis device according to claim 3 wherein the active electrode assembly further comprises:
a first electrolyte solution holding part that holds an electrolyte solution in contact with the first electrode; and
a second ion exchange membrane that selectively passes ions of a second polarity, the second ion exchange membrane being placed on a front side of the first electrolyte solution holding part,
wherein the drug holding part is placed on a front side of the second ion exchange membrane.

10. An iontophoresis device according to claim 3 further comprising a counter electrode assembly comprising:
a second electrode;
a second electrolyte solution holding part that holds an electrolyte solution in contact with the second electrode;
a third ion exchange membrane that selectively passes ions of the first polarity, the third ion exchange membrane being placed on a front side of the second electrolyte solution holding part;
a third electrolyte solution holding part that holds an electrolyte solution, the third electrolyte solution holding part being placed on a front side of the third ion exchange membrane; and
a fourth ion exchange membrane that selectively passes ions of the second polarity, the fourth ion exchange membrane being placed on a front side of the third electrolyte solution holding part.
